(19) United States
(12) Patent Application Publication
(10) Pub. No.: US 2016/0235642 A1
(43) Pub. Date: Aug. 18, 2016

(54) Melanin Binding Agents for Targeted Topical Delivery

(71) Applicant: AVON PRODUCTS, INC, New York, NY (US)

(72) Inventors: Ashley L. Howell, Oakland, NJ (US); Amitabh Bansal, Hoboken, NJ (US); Jody P. Ebanks, Nyack, NY (US)

(21) Appl. No.: 14/379,795

(22) PCT Filed: Mar. 11, 2014
(86) PCT No.: PCT/US14/23233

§ 371 Date: Aug. 20, 2014

Related U.S. Application Data
(60) Provisional application No. 61/786,787, filed on Mar. 15, 2013.

(51) Int. Cl. A61K 8/58; A61K 47/24; C07F 15/02; C07F 7/28; A61Q 19/02; A61K 33/24; A61K 33/26; C09C 1/36; C09C 1/24; C07F 7/18; A61K 9/00

(52) U.S. Cl. CPC A61K 8/585; C07F 7/1836; A61K 47/24; C07F 15/02; C07F 7/28; A61K 9/0014; A61K 33/24; A61K 33/26; C09C 1/3669; C09C 1/24; A61Q 19/02

(57) ABSTRACT

Compositions and methods are disclosed for targeted delivery of a benefit agent to areas of a human integument having melanin deposits. The compositions comprise a pyridinium compound having affinity for melanin. The compounds may be bound to a benefit agent, such as a pigment and may be topically applied to a human integument.

### RELATED APPLICATIONS

This patent application is a national stage entry of, and claims priority to, United States Patent Application Serial No. PCT/US14/23233, filed on Mar. 11, 2014; and further claims priority to U.S. Provisional Patent Application Ser. No. 61/786,787, filed on Mar. 15, 2013. Both of the aforementioned applications are herein incorporated herein in their entirety by reference.

### FIELD OF INVENTION

The present invention relates generally to compositions for topical application to skin which have a high affinity for melanin. More particularly, the present invention relates to pyridinium derivatives that have an affinity for melanin. The pyridinium derivatives are useful for targeted delivery of actives to localized deposits of melanin, including freckles, age spots, areas of hyperpigmentation and the like. The pyridinium derivatives are also useful for providing cosmetic deposits on keratin-containing integuments.

### BACKGROUND OF THE INVENTION

Several skin conditions are associated with the overproduction or unwanted production of melanin in the skin, including age spots, freckles, and liver spots. Melanin is synthesized from tyrosine and related aromatic molecules in specialized skin cells called melanocytes and is ultimately deposited in the keratinocyte skin cells after vesicular (melanosome) intercellular transfer. The most common biological melanins (e.g., those found in skin and hair) are eumelanin and pheomelanin. Eumelanin is a brown-black polymer of dihydroxyindole and dihydroxyindole carboxylic acids and their reduced forms. Pheomelanin is a red-yellow cysteine-containing polymer of benzothiazine units. There is a continuing need in the art for compositions and methods with treat areas of localized pigmentation.

It is therefore an object of the invention to provide compositions and methods which target areas of pigmentation in the skin to provide an overall aesthetic benefit. It is another object of the invention to provide compositions that adhere to keratin-containing integuments.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE INVENTION

In accordance with the foregoing objectives and others, the present invention provides methods and compositions for targeting benefit agents to pigmented areas of a human integument, including human skin. In one aspect of the invention, compositions are provided comprising, in a topically acceptable vehicle, the reaction product of a lipophilic pyridinium compound and a benefit agent. The lipophilic hydrocarbon chain typically forms a covalent bond with the ring nitrogen of a pyridine ring to form a pyridinium cation, although, in some embodiments, the lipophilic chain may be covalently bound to a ring carbon of the pyridine and the pyridine nitrogen may be covalently bound to a lower alkyl (e.g., methyl) or to hydrogen to provide a pyridinium cation. The compounds will typically have the structure of Formula (I).

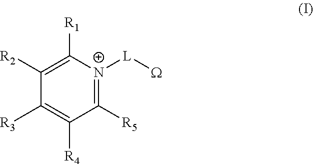

Wherein, L is a divalent C₆₋₂₄ hydrocarbon moiety including, without limitation, a group of the form —X₁—(CR*₂)ₙ—X₂—(CR*₂)ₘ—X₃—;

X₁ is selected from the group consisting of a bond (i.e., it is absent), —CH₂—, —CR*₂—, —CR*═CR*— (cis or trans), —C≡C—, —(C═O)—, —(C═O)—O—, —(C═O)—NR*—, —Si(R*)₂—, —(CR*₂)ᵢ—Si(R*)₂—, —(CR*₂)ᵢ—(Si(R*)₂)ⱼ—(O—Si(R*)₂)ₖ— (where “i” is an integer from 0-10, “j” is zero or one and “k” is an integer from 1-10);

X₂, and X₃ are independently at each occurrence a bond (i.e., it is absent), —O—, —NR*—, —S—, —CH₂—, —CR*₂—, —CR*═CR*— (cis or trans), —C≡C—, —(C═O)—, —(C═O)—O—, and —(C═O)—NR*—, —(OCH₂CH₂)_{y}—, or —(CH₂CH₂O)_{z}— (wherein “y” and “z” are independently an integer from 1 to 12), —Si(R*)₂—, —(Si(R*)₂)ⱼ—(O—Si(R*)₂)ₖ— (where “j” is zero or one and “k” is an integer from 1-10), and “n” and “m” are independently an integer from 0 to 30 (e.g., from 6 to 24, or from 8 to 20, or from 10 to 18);

Ω is selected from hydrogen, —CH₃, or a reactive group for conjugating said compound to a benefit agent, including without limitation, a nucleophilic group Nu, for example, —OH, —SH, —NH₂; —NHR*, —(C—NR*₂)═R* (enamine); a leaving group L, including, without limitation, chloro, bromo, iodo, mesyl, and tosyl; a reactive group of the form —CR*═CR*₂, —C≡CH, —CR*(O)CR* (epoxide), —Si(OR*)₃ (alkoxysilane), —Si(R*)₂H (silane), —(Si(R*)₂)ⱼ—(O—Si(R*)₂)ₖ—Si(OR*)₃, —(Si(R*)₂)ⱼ—(O—Si(R*)₂)ₖ—Si(R*)₂H, —CO₂H; —CO₂R*; —C(═O)Cl (acyl halide), —CH₂O(C═O)CH₂(C═O)CH₃ (beta-keto ester), —C(O)—O—(N(—(C═O)—R*)₂) (e.g., succinimide ester or pthalamide ester), —SO₂Cl, —NCS, —NCO, —N₃, —N₂⁺Cl⁻; or a group R (as defined below);

R₁-R₅ are independently selected, at each occurrence, from a group R; wherein R is selected from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —SH; —SR*; —C≡N, —NC; —(C═O)—R*; —CHO; —CO₂H; —CO₂⁻; —CO₂R*; —C(═O)Cl, —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃⁻; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; —CH₃, —CR*═CR*₂, —C≡CH, —Si(OR*)₃, —Si(R*)₂H, C₁-C₁₂ perfluoroalkyl; an aliphatic C₁-C₁₂ hydrocarbon radical; a C₁-C₁₂ aromatic hydrocarbon radical; or a C₁-C₁₂ heteroaryl radical;

where R* is independently at each occurrence hydrogen or a straight chained, branched, or cyclic C₁-C₂₀ hydrocarbon radical, which may be saturated, partially saturated, or aromatic, each of which may be optionally substituted with 1-6 heteroatoms selected from nitrogen, oxygen, sulfur, or halogen, and wherein any two adjacent R* groups may together form a 3-8 membered aromatic, partially unsaturated, or saturated ring;

In one implementation L is a (preferably lipophilic) C₆₋₂₄ hydrocarbon moiety; including, without limitation C₆₋₂₄ linear, branched, cyclic (or combinations thereof) hydrocarbon moieties that are saturated, partially or fully unsaturated, or aromatic, and which include halogenated and perhalogenated (e.g., perfluorinated) derivatives, and which may optionally comprise from 1-10 heteroatoms selected from the group consisting of oxygen, nitrogen, and sulfur; including oxo, oxa, and thioether derivatives. In some embodiments, L will further be defined as having an octanol-water partition coefficient, Log(K_{ow}) of the corresponding alcohol HO-L-Ω or of the corresponding alcohol HO-L-CH₃ generally greater than 1 or between 1 and about 10, typically greater than about 2 (e.g., between about 2 and about 10), and more typically, greater than about 3 (e.g., between about 3 and about 10), about 4 (e.g., between about 3 and about 10), about 5 (e.g., between about 5 and about 10), about 6 (e.g., between about 6 and about 10), about 7 (e.g., between about 7 and about 10), or about 8 (e.g., between about 8 and about 10), including embodiments where the Log(K_{ow}) of the corresponding alcohol HO-L-Ω or HO-L-CH₃ is between about 6 and about 8, including an embodiment of about 7.

In another aspect, a surface-functionalized particle is provided comprising the reaction product between a compound of Formula (I) and a particulate composition (e.g., an inorganic oxide particle) having a plurality of reactive functional groups (e.g., —OH or —O⁻) on the surface thereof. In one implementation, the compound of Formula (I) will comprise a lipophilic chain L (e.g., C₈₋₁₈ alkyl or alkenyl) terminated with a functional group Ω that is capable of forming a covalent bond with at least some of said plurality of reactive functional groups (e.g., —OH or —O⁻). In one implementation, Ω will be a trialkoxysilane of the form —Si(OR*)₃, where R* is a lower alkyl, such as methyl, ethyl, propyl, butyl, and mixtures thereof. In one embodiment, R* will be methyl or ethyl at each occurence. The group —Si(OR*)₃ is capable of forming a covalent siloxane bond with free hydroyl groups on the surface of a particle, such a laked pigment (for example, red 7 Ca lake) or an inorganic oxide (e.g., silica and metal oxides, such as iron oxide, titanium oxide, zinc oxide, and the like). The degree of surface functionalization may be from about 0.01% to about 50% by weight or more, typically from about 0.1% to about 10% by weight, or from about 0.5% to about 5% by weight.

In one embodiment, the surface functionalized pigment is the reaction product between a compound of Formula (I) where R₁-R₅ are hydrogen, chain L is a C₈₋₁₆ alkyl group (e.g., C₈, C₉, C₁₀, C₁₁, C₁₂, C₁₃, C₁₄, C₁₅, or C₁₆) characterized by an octanol-water partition coefficient Log(K_{ow}) of the corresponding alcohol HO-L-CH₃ between about 4 and about 10, and Ω is a trialkoxysilane of the form —Si(OCH₃)₃ or —Si(OCH₂CH₃)₃.

Generally, the surface-functionalized pigments comprise pigment particles (e.g., inorganic oxides having median particle size between about 5 nm and about 250 microns) having bonded (e.g., covalently) to the surface thereof, a plurality of lipophilic pyridinium compounds. The lipophilic pyridinium compounds may comprise a lipophilic hydrocarbon chain which may be attached at one end, directly or through a functional group, to the surface of the pigment. A pyridine ring is attached to or near a distal end of the liphophilic hydrocarbon chain. The lipophilic hydrocarbon chain typically forms a covalent bond with the ring nitrogen of a pyridine ring to form a pyridinium cation. Although, in some embodiments, the lipophilic chain may be covalently bound to a ring carbon and the pyridine nitrogen may be covalently bound to a lower alkyl (e.g., methyl) or a hydrogen to provide a pyridinium cation. The lipophilic hydrocarbon chain is typically attached to the surface of the pigment particle through a silane linkage.

In a further aspect of the invention compounds of Formula (II) are provided:

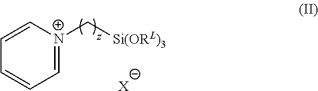

wherein, “z” is 6-18 (or 8-16 or 10-12), R^{L} is independently at each occurrence lower alkyl (e.g., methyl or ethyl), and X is a topically acceptable anion (e.g., chloride, bromide, carboxylate, sulfate, etc.).

In another aspect, methods of treating keratinous surfaces (e.g., skin, hair, nails) are provided, comprising applying a composition comprising a conjugate of a compound of Formula (I) and a benefit agent to the keratinous surface. The composition may further include a topically acceptable vehicle and may be applied at least once weekly, more typically at least once daily, for a period sufficient to show improvement in the overall aesthetic appearance of the keratinous substrate. The duration of treatment may be, without limitation, single use, daily for at least five days, or daily for at least one or two weeks, or longer.

These and other aspects of the present invention will become apparent to those skilled in the art after a reading of the following detailed description of the invention, including the appended claims.

### BRIEF DESCRIPTION OF THE FIGURES

Affinity of pyridinium compounds for melanin in Melanoderm Mel-B (high concentration of melanin) and Melanoderm Mel-C (low concentration of melanin) tissue models.

### DETAILED DESCRIPTION

All amounts provided in terms of weight percentage are relative to the entire composition unless otherwise stated. Unless otherwise provided, the term “alkyl” is intended to embrace straight-chained, branched, or cyclic hydrocarbons, particularly those having from one to 20 carbon atoms, and more particularly C₁₋₁₈ hydrocarbons. The term “lower alkyl” includes, without limitation methyl, ethyl, propyl, isopropyl, butyl, n-butyl, iso-butyl, tert-butyl, pentyl, neo-pentyl, amyl, and hexyl, including cyclized analogues thereof. Unless otherwise provided, the term “alkenyl” is intended to embrace straight-chained, branched, or cyclic hydrocarbons, particularly those having from one to 20 carbon atoms, and more particularly C₁₋₁₈ hydrocarbons, which may include one, two, three, or more unsaturated carbon-carbon bonds, which may be in the E or Z configuration.

As used herein, the term “keratinous surface” refers to keratin-containing portions of the human integumentary system, which includes, but is not limited to, skin, lips, hair (including eyebrows and eyelashes), and nails (toenails, fingernails, cuticles, etc.) of mammalians, preferably humans. A “keratin fiber” includes hair of the scalp, eyelashes, eyebrows, facial hair, and body hair such as hair of the arms, legs, etc. Keratin fibers are not limited to humans and also include any keratin fibers from a mammal, such as, for example, pet hair and mammalian fur.

The term “benefit agent” is used to describe cosmetic, dermatological, personal care, or pharmaceutical agents that provide aesthetic or therapeutic enhancement to a keratinous substrate, such as, human skin, hair or nails. Preferably, the benefit agent has one or more functional groups capable of forming a covalent linkage to either the group Ω (or a portion or fragment thereof) or to the group L (e.g., if Ω is a leaving group). For example, the functional group may be a hydroxyl functional group that forms a reaction product (e.g., an ester or siloxane bond) with a compound of Formula I.

The compounds of the invention will generally have the structure shown of Formula (I).

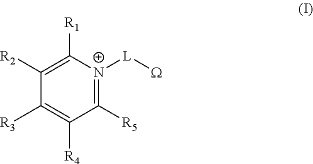

Wherein, L is a hydrogen or is a divalent C₄₋₃₂ or C₅₋₂₂ or C₆₋₁₆ or C₈₋₁₄ or C₁₀₋₁₂ hydrocarbon moiety including, without limitation, branched, cyclic, or straight-chained aliphatic hydrocarbon radicals, such as alkyl, alkenyl, and alkynyl groups, each of which may be optionally substituted with 1-20 heteroatoms selected from Si, O, S, N, and halogen (e.g., fluorine), including substitution with from 1 to 6 independently selected groups R (defined below). In one embodiment, L is a straight chained C₄₋₂₂ aliphatic hydrocarbon, such as an alkyl or alkenyl (having one, two, three, or more trans or cis double bonds). In one embodiment, L is a straight chained C₄₋₃₂ or C₅₋₂₂ or C₆₋₁₆ alkyl group. In one embodiment, L is a straight chained C₄₋₃₂ or C₅₋₂₂ or C₆₋₁₆ alkenyl group. In one embodiment, the alkenyl group has one double bond, typically in a trans configuration. The double bond, if present, may be, for example, adjacent to the nitrogen atom of the pyridinium ring.

In one embodiment, L is a divalent C₄₋₃₂ hydrocarbon moiety of the form —X₁—(CR*₂)ₙ—X₂—(CR*₂)ₘ—X₃—, where X₁ is selected from the group consisting of a bond (i.e., it is absent), —CH₂—, —CR*₂—, —CR*═CR*— (cis or trans), —C≡C—, —(C═O)—, —(C═O)—O—, —(C═O)—NR*—, —Si(R*)₂—, —(CR*₂)ᵢ—Si(R*)₂—, —(CR*₂)ᵢ—(Si(R*)₂)ⱼ—(O—Si(R*)₂)ₖ— (where “i” is an integer from 0-10, “j” is zero or one and “k” is an integer from 1-10); and X₂, and X₃ are independently at each occurrence a bond (i.e., it is absent), —O—, —NR*—, —S—, —CH₂—, —CR*₂—, —CR*═CR*— (cis or trans), —C≡C—, —(C═O)—, —(C═O)—O—, and —(C═O)—NR*—, —(OCH₂CH₂)_{y}—, or —(CH₂CH₂O)_{z}— (wherein “y” and “z” are independently an integer from 1 to 12), —Si(R*)₂—, —(Si(R*)₂)ⱼ—(O—Si(R*)₂)ₖ— (where “j” is zero or one and “k” is an integer from 1-10), and “n” and “m” are independently an integer from 0 to 30 (e.g., from 6 to 24, or from 8 to 20, or from 10 to 18);

In one embodiment, L is a straight chained C₅, C₆, C₇, C₈, C₉, C₁₀, C₁₁, C₁₂, C₁₃, C₁₄, C₁₅, or C₁₆ alkyl group. In one embodiment, L is a straight chained C₅, C₆, C₇, C₈, C₉, C₁₀, C₁₁, C₁₂, C₁₃, C₁₄, C₁₅, or C₁₆ alkenyl group, and in each case, the alkenyl radical may comprise a single double bond, for example adjacent to the nitrogen of the pyridinium ring. In one embodiment, L is a straight chained C₅, C₆, C₇, C₈, C₉, C₁₀, C₁₁, C₁₂, C₁₃, C₁₄, C₁₅, or C₁₆ alkyl group which includes a single oxygen atom (ether) or sulfur atom (thioether) in the chain. In one embodiment, L has the form —R^{a}—O—R^{b}— or —R^{a}—S—R^{b}— where R^{a} and R^{b} are independently C₁₋₁₂ or C₂₋₈ straight chain alkyl groups. In one embodiment, L has the form —R^{a}—Si(CH₃)₂—R^{b}— where R^{a} and R^{b} are independently C₁₋₁₂ or C₂₋₈ straight chain alkyl groups. In another embodiment, L has the form —R^{a}—Si(CH₃)₂—O—Si(CH₃)₂—R^{b}—, where R^{a} and R^{b} are independently C₁₋₁₂ or C₂₋₈ straight chain alkyl groups. In another embodiment, L has the form —R^{a}—Si(CH₃)₂—(O—Si(CH₃)₂)k-R^{b}—, where R^{a} and R^{b} are independently C₁₋₁₂ or C₂₋₈ straight chain alkyl groups and “k” is an integer from 1-12 (or from 1-6 or from 1-3).

In one embodiment, Ω is selected from hydrogen, —CH₃, or a reactive group for conjugating said compound to a benefit agent, including without limitation, a nucleophilic group Nu, for example, —OH, —SH, —NH₂; —NHR*, —(C—NR*₂)═R* (enamine); a leaving group L, including, without limitation, chloro, bromo, iodo, mesyl, and tosyl; a reactive group of the form —CR*═CR*₂, —C≡CH, —CR*(O)CR* (epoxide), —Si(OR*)₃ (alkoxysilane), —Si(R*)₂H (silane), —(Si(R*)₂)ⱼ—(O—Si(R*)₂)ₖ—Si(OR*)₃, —(Si(R*)₂)ⱼ—(O—Si(R*)₂)ₖ—Si(R*)₂H, —CO₂H; —CO₂R*; —C(═O)Cl (acyl halide), —CH₂O(C═O)CH₂(C═O)CH₃ (beta-keto ester), —C(O)—O—(N(—(C═O)—R*)₂) (e.g., succinimide ester or pthalamide ester), —SO₂Cl, —NCS, —NCO, —N₃, —N₂⁺Cl⁻; or a group R (as defined below). In one embodiment, Ω is not hydrogen. In one embodiment, Ω is not —CH₃. One skilled in the art will recognize that various chemistries are available for conjugating the compound of Formula (I) to a given benefit molecule and can select Ω to provide an appropriate bond with a given benefit agent. The bond may be, without limitation, covalent, ionic, or a hydrogen bond. Typically, the bond will be a covalent bond.

In one embodiment, Ω is a trialkoxysilane of the form —Si(OR*)₃ where R* is typically lower alkyl, an most notably methyl and/or ethyl. Mixed alkoxysilanes are also contemplated. In one embodiment, Ω is —Si(OCH₃)₃. In another embodiment, Ω is —Si(OCH₂CH₃)₃.

R₁-R₅ are independently selected, at each occurrence, from a group R; wherein R is selected from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —SH; —SR*; —C≡N, —NC; —(C═O)—R*; —CHO; —CO₂H; —CO₂⁻; —CO₂R*; —C(═O)Cl, —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃⁻; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; —CH₃, —CR*═CR*₂, —C≡CH, —Si(OR*)₃, —Si(R*)₂H, C₁-C₁₂ perfluoroalkyl; an aliphatic C₁-C₁₂ hydrocarbon radical; a C₁-C₁₂ aromatic hydrocarbon radical; or a C₁-C₁₂ heteroaryl radical.

In one, embodiment, R₁-R₅ are each hydrogen. In one embodiment at least one, at least two, at least three, or at least four of the groups R₁-R₅ are hydrogen. In one embodiment, each of R₁-R₅ is hydrogen except R₃.

R* is independently at each occurrence hydrogen or a straight chained, branched, or cyclic C₁-C₂₀ hydrocarbon radical, which may be saturated, partially saturated, or aromatic, each of which may be optionally substituted with 1-6 heteroatoms selected from nitrogen, oxygen, sulfur, or halogen, and wherein any two adjacent R* groups may together form a 3-8 membered aromatic, partially unsaturated, or saturated ring. R* may be, for example, lower alkyl, including methyl and ethyl. R* may be, for example, trifluoromethyl. R* may be, for example, alkoxyl, such as methoxyl.

In some embodiments, L will further be defined as having an octanol-water partition coefficient, expressed as Log(K_{ow}), of the corresponding alcohol HO-L-Ω or of the corresponding alcohol HO-L-CH₃ generally greater than 1 or between 1 and about 10, typically greater than about 2 (e.g., between about 2 and about 10), and more typically, greater than about 3 (e.g., between about 3 and about 10), about 4 (e.g., between about 3 and about 10), about 5 (e.g., between about 5 and about 10), about 6 (e.g., between about 6 and about 10), about 7 (e.g., between about 7 and about 10), or about 8 (e.g., between about 8 and about 10), including embodiments where the Log(K_{ow}) of the corresponding alcohol HO-L-Ω or HO-L-CH₃ is between about 6-8, including an embodiment of about 5-7. The octanol-water partition coefficient may be determined by the “shake method.” See J. Sangster, “Octanol-water Partition Coefficients of Simple Organic Compounds,” J. Phys. Ref. Data 18(3), 1111-1129 (1989).

In a particular embodiment, L is a C₅₋₁₆ aliphatic hydrocarbon having a Log(K_{ow}), of the corresponding alcohol HO-L-CH₃ of about 1.5 to about 7, and Ω is a trialkoxysilane of the form —Si(OCH₃)₃ or —Si(OCH₂CH₃)₃. In this embodiment, R₁-R₅ are typically (but not necessarily) hydrogen.

The compound of Formula (I) may include a counterion to the quaternized nitrogen of the pyridinium ring. The counterion may be any topically acceptable anion, including without limitation chloride, bromide, iodide, sulfate, phosphate, acetate, citrate, mesylate, tosylate, besylate, and the like. In one embodiment, the compound of formula (I) is in the form of the chloride (Cl⁻) salt.

Only those compounds of Formula (I) and salts thereof that are safe for contact with human integuments are considered appropriate for use in accordance with the invention.

The compounds of Formula (I) will find particular use in delivering benefit agents to a keratinous surface. Due to the high affinity of the compounds for melanin, it is contemplated that they will be useful for preferentially delivering benefit agents to areas of localized pigmentation or hyperpigmentation, such as freckles, age spots, sun spots, liver spots, etc. In one embodiment, a conjugate is formed as the reaction product of compound of Formula (I) and a benefit agent. In one embodiment, the benefit agent is in particulate form. Suitable particulates include, without limitation, organic pigments, laked pigments and inorganic oxide particles (e.g., silica or metal oxides where the metal is preferably selected from among magnesium, calcium, aluminum, iron, titanium, zirconium, chromium, manganese, cobalt, cerium, nickel, zinc and composites and mixtures thereof).

Exemplary inorganic pigments include, but are not limited to, inorganic oxides and hydroxides such as magnesium oxide, magnesium hydroxide, calcium oxide, calcium hydroxides, aluminum oxide, aluminum hydroxide, iron oxides (α-Fe₂O₃, y-Fe₂O₃, Fe₃O₄, FeO) and iron hydroxides including red iron oxide, yellow iron oxide and black iron oxide, titanium dioxide, titanium lower oxides, zirconium oxides, chromium oxides, chromium hydroxides, manganese oxides, manganese hydroxides, cobalt oxides, cobalt hydroxides, cerium oxides, cerium hydroxides, nickel oxides, nickel hydroxides, zinc oxides and zinc hydroxides and composite oxides and composite hydroxides such as iron titanate, cobalt titanate and cobalt aluminate and the like. Preferably, the inorganic oxide particles may be selected from silica, alumina, zinc oxide, iron oxide and titanium dioxide particles, and mixtures thereof. Although silica, alumina, iron oxide, zinc oxide, and titanium dioxide particles are preferred, one of skill would understand that any particle which is capable of being functionalized by reaction with the compound may be used.

In some embodiments, the benefit agent is a particle. In one embodiment, the minimum particle size is 5 nm or 10 nm. The particle size may range upwards to 1 micron, 10 microns, 100 microns, 500 microns and above.

In one embodiment, the benefit agent may be an agent that combats pigmentation or hyperpigmentation, including tyrosinase inhibitors and/or melanosome transfer inhibitors, as well as agents that accelerate epidermal turnover such as alpha-hydroxy acids; salicylic acid; linoleic acid; retinoids (e.g., retinoic acid), etc; Centaureidin; methylophiopogonanone B; niacinamide; PAR-2 inhibitors; lectins; neoglycoproteins; hydroquinone; hydroquinone-beta-D-glucopyranoside; retinoids (e.g., retinoic acid); tretinoin; azelaic acid; Kojic acid (5-hydroxy-4-pyran-4-one-2-methyl); Mequinol (4-hydroxyanisole); soy protein and other serine protease inhibitors; paper mulberry extract; Glabridin (licorice extract); Arctostaphylos patula and Arctostaphylos viscida extracts; Glycyrrhiza glabra and its derivatives; Chlorella vulgaris extract; Magnesium-L-ascorbyl-2-phosphate (MAP); 4-Isopropylcatechol; Aleosin; N-acetyl-4-S-cysteaminylphenol and N-propionyl-4-S-cysteaminylphenol; N-acetyl glucosamine; and Tranexamic acid (trans-4-aminomethylcyclohexanecarboxylic acid); arbutin and derivatives thereof (e.g., deoxyarbutin), bearberry extract, ascorbic acid and/or its derivatives, perilla extract (e.g., in U.S. Pat. No. 5,980,904 and Japanese Publications Nos. 07025742, 07187989, 10265322, 2001163759, and 2001181173, incorporated herein by reference), coconut fruit extract (Japanese Patent No. 2896815B2, incorporated by reference herein), and calcium influx inhibitors, to name a few. Any of the tyrosine inhibitors disclosed in KR 2005095167; JP 2003252743; and JP 61260009, incorporated by reference herein, may be included, in some embodiments. Other skin lighteners include extracts of Butea frondosa, Naringi crenulata, Stenoloma chusana, Azadirachta indica, Glycyrrhiza glabra linn., Morinda citrifolia, tomato glycolipid, ascorbyl glucoside, vitamin C, retinol and/or its derivatives, rumex crispus extract, milk proteins including hydrolyzed milk proteins, N,N,S-tris(carboxymethyl)cysteamine, oleanolic acids, placenta extract, saxifragia sarmentosa, juniperic acid, ligusticum chiangxiong hort., asmunda japonica thunb., stellaria medica (L.) cyr., sedum sarmentosum bunge, ligusticum lucidum Ait., ilex purpurea hassk, emblica, apigenin, ascorbyl palmitol, carruba C. borealis s, hesperitin, inabata C. borealis, isoliquirtigenin, kaempherol-7-neohesperidose, L-mimosine, luteolin, oil-soluble licorice extract P-T(40), oxa acid, phenyl isothiocyanate, cococin, silymarin, T4CA, teterahydro curcumin, unitrienol, ursolic-oleanolic acid, UVA/URSI, or any combinations thereof.

Other suitable benefit agents include those agents that provide a prophylactic or therapeutic benefit to skin. Particular mention may be made of alpha-hydroxy acids, beta hydroxyl acids, ascorbic acid or Vitamin C and derivatives thereof (e.g., C₁-C₈ esters thereof); retinoids such as retinol (Vitamin A) and the esters thereof (e.g., C₁-C₈ esters, such as palmitate), retinoic acid and the derivatives thereof, hyaluronic acid, chemical sunscreens useful in the cosmetic field including any UVA and UVB filter useful in the cosmetic field including mixtures thereof and blends with physical filters including, but not limited to metal oxide particles such as titanium oxides and/or zinc oxides. Additional benefit agents include botanicals, thiodipropionic acid (TDPA) and esters thereof; retinoids (e.g., all-trans retinoic acid, 9-cis retinoic acid, phytanic acid and others); hydroxy acids (including alpha-hydroxyacids and beta-hydroxyacids), salicylic acid and salicylates; exfoliating agents (e.g., glycolic acid, 3,6,9-trioxaundecanedioic acid, etc.), depigmenting agents (e.g., hydroquinone, kojic acid, etc.) estrogen synthetase stimulating compounds (e.g., caffeine and derivatives); compounds capable of inhibiting 5 alpha-reductase activity (e.g., linolenic acid, linoleic acid, finasteride, and mixtures thereof); antioxidants (e.g., thiodipropionic acid, vitamin E, etc.), barrier function enhancing agents (e.g., ceramides, glycerides, cholesterol and its esters, alpha-hydroxy and omega-hydroxy fatty acids and esters thereof, etc.); collagenase inhibitors; and elastase inhibitors; to name a few.

Other benefit agents that may be functionalized as described herein, in accordance with the present invention, may further include, soft focus ingredients, colorants (e.g., pigments, lakes or dyes), anti-acne agents, sunscreens, self-tanning ingredients, anti-inflammatory agents, antiseptic agents, insect repellants, anti-bacterials, anti-fungals, anti-virals, anti-yeast agents, age spot treatments, anti-oxidants, moisturizing agents, antiseptic agents, analgesics, antidandruff and antiseborrhetic agents, hyperkeratolytics, antipsoriatic agents, skin lightening agents, depigmenting agents, wound healing agents, burn treatments, tanning agents, hair treatment agents, hair growth products, wart removers, hormones, antipyretics, agents for lupus, multiform erythema, photo allergic and photo toxic reaction and atopic dermatitis or a body personal care composition, such as an antiperspirant or deodorant.

The foregoing benefit agents (including the depigmenting agents) may be conjugated to the compounds of Formula (I) through chemistries that will be readily apparent to those of skill in the art. Optionally, one or more of these benefit agents may be included as an additional component of the composition, i.e., as a separate ingredient from the conjugate composition. These additional benefit agents will typically be present, if at all, in amounts between about 0.001% and about 10% by weight of the composition. In one embodiment, the additional benefit agents may be present in amounts between about 0.001% and about 20% by weight of the composition. In another embodiment, the additional benefit agents may be present in amounts between about 0.001% and about 30% by weight of the composition.

The compositions will usually comprise a compound of Formula (I), or a conjugate of a compound of formula (I) and a benefit agent, in an amount from about 0.1% to about 50% by weight of the total composition, more typically from about 0.5% to about 25% by weight or from about 1% to about 10% by weight of the total composition.

The compositions of the invention will typically include a cosmetically or dermatologically acceptable vehicle, which may be in the form of, for example, a serum, a cream, a lotion, a gel, or a stick, and may comprise an emulsion (e.g., water-in-oil, oil-in-water, water-in-silicone, silicone-in-water, polyol-in-silicone, silicone-in-polyol emulsion, etc.), or may comprise an aqueous or ethanolic vehicle, silicone (e.g., cyclomethicone, dimethicone, etc.), hydrocarbon (e.g., petrolatum, isododecane, etc.), ester oil (isopropyl myristate, myristyl myristate, or the like. The vehicle may be anhydrous and may comprise oils, such as dimethicones, hydrocarbons (e.g., isododecane), petrolatum, ester oils, and the like. The vehicle may further comprise an emulsifier, gelling agent, structuring agent, rheology modifier (e.g., a thickener), film former, or the like. The vehicle may comprise from about 5% to about 95% by weight of the composition, in one embodiment from about 10% to about 90%, in one embodiment from about 20% to about 80%, in one embodiment from about 50% to about 99% by weight of the composition.

The vehicle may comprise an aqueous phase, an oil phase, an alcohol, a silicone phase or mixtures thereof, and may be in the form of an emulsion. Non-limiting examples of suitable emulsions include water-in-oil emulsions, oil-in-water emulsions, silicone-in-water emulsions, water-in-silicone emulsions, polyol-in-silicone emulsions, silicone-in-polyol emulsions, polyol-in-oil emulsions, oil-in-polyol emulsions, wax-in-water emulsions, water-oil-water triple emulsions or the like. The emulsion may include an emulsifier, such as a nonionic, anionic or amphoteric surfactant, or a gelling agent.

The vehicle may comprise water; vegetable oils; mineral oils; esters such as octyl palmitate, myristyl myristate, isopropyl myristate, and isopropyl palmitate; ethers such as dicapryl ether and dimethyl isosorbide; alcohols such as ethanol and isopropanol; fatty alcohols such as cetyl alcohol, cetearyl alcohol, and stearyl alcohol; volatile silicones such as cyclomethicones, silicone oils like dimethicone, amodimethicones, and dimethiconol; hydrocarbons such as mineral oil, petrolatum, and isoparaffins such as isooctane, isododecane (IDD), isohexadecane, and isoeicosane; and (hydrogentated) polyolefins such as polyisobutene; polyols such as propylene glycol, glycerin, butylene glycol, pentylene glycol and hexylene glycol; liposomes; waxes (animal, vegetable, or synthetic); or any combinations or mixtures of the foregoing.

The compositions may include natural or synthetic film-forming polymers. Suitable polymeric film formers include polyolefins, silicone polymers (e.g., dimethicones, dimethiconols, amodimethicones, etc.), (meth)acrylates, alkyl (meth)acrylates, polyurethanes, fluoropolymers, and silicone acrylates such as acrylates/dimethicone copolymers. In some embodiments, it may be desirable to add a hydrophilic or water-soluble film former (e.g., cellulosics, polysaccharides, polyquaterniums (such as polyquatemium-37 (INCI), etc.) to the composition to improve spreading, emulsion stability, aesthetic look and feel, etc.

The compositions may further include an emulsifier. The amount of emulsifier will typically be from about 0.001 to about 10% by weight, but preferably will range from about 0.01 to about 5% by weight, and most preferably about 0.1 to about 1% by weight, based upon the total weight of the composition. The emulsifier may be ionic, zwitterionic, or nonionic. Suitable emulsifiers include those of the polyethoxylated type (e.g., polyoxyethylene ethers or esters), polydiorganosiloxane-polyoxyalkylene block copolymers (e.g., dimethicone copolyol), Steareth-20, Steareth-21, fatty alcohols (e.g., Cetearyl Alcohol), Polyoxethylene sorbitan fatty acid esters (i.e., polysorbates), and Hydrogenated Castor Oil, to name a few. Additional emulsifiers are provided in the INCI Ingredient Dictionary and Handbook 11th Edition 2006, the disclosure of which is hereby incorporated by reference.

The composition may also comprise humectants such as polyols (e.g., glycols), including without limitation, glycerin, propylene glycol, ethoxydiglycol, butylene glycol, pentylene glycol, hexylene glycol, caprylyl glycol, and the like. These will typically be added in amount from about 0.001 to about 5% by weight.

In another embodiment, the topical compositions of the present invention may also include one or more of the following: a skin penetration enhancer; an emollient, such as isopropyl myristate, petrolatum, volatile or non-volatile silicones oils (e.g., methicone, dimethicone), ester oils, mineral oils, and fatty acid esters; a humectant, such as glycerin, hexylene glycol or caprylyl glycol; a skin plumper, such as palmitoyl oligopeptide, collagen, collagen and/or glycosaminoglycan (GAG) enhancing agents; a sunscreen, such as avobenzone; an exfoliating agent; and an antioxidant.

The compositions of the invention may optionally include additional skin benefit agents such as emollients (dimethicone oils, ester oils, or hydrocarbon oils), humectants (e.g., polyols, including propylene glycol, glycerin, etc.), antioxidants (e.g., BHT, ascorbic acid, sodium ascorbate, ascorbyl palmitate, beta-carotene, etc.), vitamins (e.g., tocopherol, tocopheryl acetate, etc.), alpha-hydroxy acids (e.g., glycolic acid), beta-hydroxy acids (e.g., salicylic acid), retinoids (e.g., retinoic acid, all-trans-retinoic acid, retinaldehyde, retinol, and retinyl esters such as acetates or palmitates), other anti-aging ingredients (e.g., collagen stimulators), as well as additional depigmenting agents.

The compositions of the invention will typically have a pH ranging from 2-12, more typically from 2 to 10. In some embodiments, the compositions will have a pH ranging from about 3 to about 7, or from about 3.5 to about 6.5. The pH may be adjusted using conventional acid or base pH adjusters including, without limitation, citric acid, mineral acids, ethanolamine and other amines, ammonia, sodium hydroxide, etc.

In addition to the foregoing, the compositions according to the invention may comprise pigments, pearlescents, and/or colorants. Inorganic pigments include without limitation titanium dioxide, zinc oxide, iron oxides, chromium oxide, ferric blue, and mica; organic pigments include barium, strontium, calcium or aluminium lakes, ultramarines, and carbon black; colorants include without limitation D&C Green #3, D&C Yellow #5, and D&C Blue #1. Pigments and/or colorants may be coated or surface treated with one or more compatibilizers to aid in dispersion in the solvent, and may include organic lakes. Preferred pigments and/or colorants are those surface treated to render them hydrophobic.

The composition typically comprises a preservative or anti-microbial agent, for example, methylchloroisothiazolinone, methylisothiazolinone, methylparaben, propylparaben, phenoxyethanol, or caprylyl glycol.

Other additives include: vitamins, such as tocopherol and ascorbic acid; vitamin derivatives such as ascorbyl monopalmitate, tocopheryl acetate, and Vitamin E palmitate; thickeners such as hydroxyalkyl cellulose, carboxymethylcellulose, carbombers, and vegetable gums such as xanthan gum; gelling agents, such as ester-terminated polyester amides; structuring agents; metal chelating agents such as EDTA or salts thereof; pigments; colorants; and pH adjusters (citric acid, ethanolamine, sodium hydroxide, etc.). The composition may optionally comprise other components known to those skilled in the art including, but not limited to, film formers, moisturizers, minerals, viscosity and/or rheology modifiers, anti-acne agents, insect repellents, skin cooling compounds, skin protectants, lubricants, fragrances, preservatives, stabilizers, and mixtures thereof. In addition to the foregoing, the cosmetic compositions of the invention may contain any other compound for the treatment of skin disorders.

A sunscreen may be included to protect the skin from damaging ultraviolet rays. In an illustrative embodiment of the present disclosure, the sunscreen provides both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, octocrylene, titanium dioxide, zinc oxide, or any mixtures thereof. The sunscreen may be present from about 1 wt % to about 30 wt % of the total weight of the composition.

The composition may be formulated in a variety of product forms, such as, for example, an emulsion, lotion, cream, serum, spray, aerosol, cake, ointment, essence, gel, paste, patch, pencil, towelette, mask, stick, foam, elixir, concentrate, and the like, particularly for topical administration. The composition is typically formulated as an emulsion, lotion, cream, ointment, serum or gel. The compositions can be formulated into liposomes which can comprise other additives or substances, and/or which can be modified to more specifically reach or remain at a site following administration. Another embodiment of the present disclosure is directed to the delivery of the described compositions by the use of targeted delivery systems, for example, liposomes, microspheres (see, e.g., U.S. Pat. No. 5,770,222 to Unger et al.), and the like, so that the components and/or active constituents can more readily reach and affect the subcutaneous layer of the area of application, e.g., face or neck, or the other area of the skin.

The compositions are typically applied for a time sufficient to improve the overall appearance of the integument (e.g., skin, hair, nails, etc.). This typically entails single use, or application daily for at least three days, at least five days, at least, one week, or at least two weeks, although it is expected that results may be seen after one use.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, §201(i).

### Example 1

Compounds of Formula (II), where “z” is eleven (11) and X is chloride, were reacted with yellow iron oxide and titanium dioxide to provide surface-functionalized yellow iron oxide and titanium dioxide pigments.

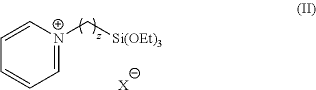

In one embodiment, where “z” is eleven (11) and X is chloride, the compound was reacted with yellow iron oxide and titanium dioxide to provide surface-functionalized yellow iron oxide and titanium dioxide pigments.

The surface-functionalized yellow iron oxide and titanium dioxide pigments were mixed with an aqueous solution of synthetic melanin. In the case of the yellow iron oxide, it was found that the melanin and surface-functionalized pigment precipitated from solution and settled out. In contrast, when the untreated yellow iron oxide was mixed with synthetic melanin, the pigment settled from solution but the melanin remained suspended. Likewise, when cetylpyridinium chloride and untreated yellow iron oxides were mixed with the solution of synthetic melanin, the untreated yellow iron oxide pigment settled from solution, but the ceytlpyridinium chloride and melanin remained suspended. This indicates that the pyridinium compound binds to melanin, and in the case of the surface-treated pigments, the melanin is pulled from solution by the pigments. In the case of titanium dioxide, it was found that both the melanin and the surface-functionalized pigments stay suspended in solution. In contrast, when the untreated titanium dioxide was mixed with synthetic melanin, the titanium dioxide settles out of solution, whereas the melanin stays suspended in solution. In both cases, the surface-functionalized pigment associates (binds) with the melanin, and the melanin remains wherever the surface functionalized pigment is located.

### Example 2

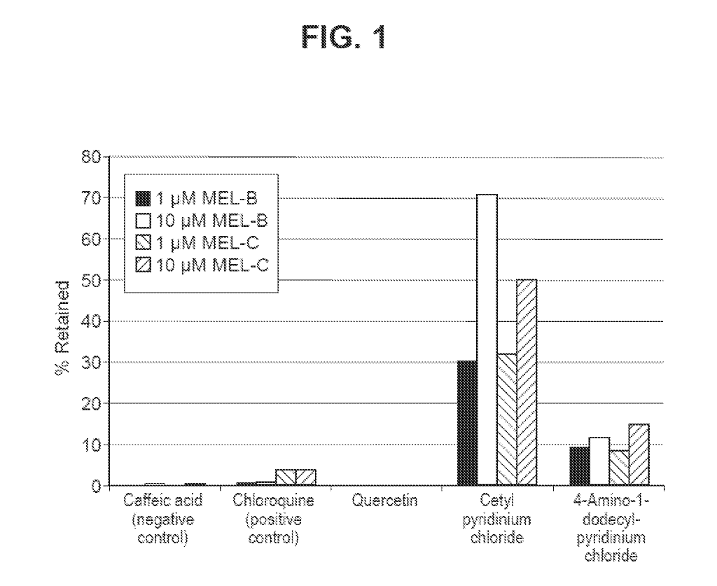

Melanoderm Mel-B (high concentration of melanin) and Melanoderm Mel-C (low concentration of melanin) tissue models were used to determine the affinity of pyridinium compounds for melanin. Chloroquine served as a positive control and caffeic acid was a negative control for melanin binding. Cetyl pyridinium chloride and 4-amino-1-dodecyl-pyridinium chlorides were test compounds. Test materials were topically applied at 0, 24 and 48 hours and tested at 1 μM and 10 μM concentration in water. Media samples were collected at 6, 24, 48, and 72 hours and tissue were lysed at 72 hours. The results are shown in FIG. 1. As shown, both the cetyl pyridinium chloride and 4-amino-1-dodecyl-pyridinium chloride exhibit strong binding affinity for the 3-D tissue samples, with the data reflecting increased melanin binding seen when comparing the 10 μM to the 1 μM concentrations of cetyl pyridinium chloride as well as comparing the 10 μM to the 1 μM concentrations of the 4-amino-1-dodecyl-pyridinium chloride. Each outperformed the positive control.

### Example 3

Free synthetic melanin was incubated with compounds of interest in order to determine relative binding affinity of various compounds of interest to melanin. Free synthetic melanin (50 μg/mL) and compounds of interest at 10 μM concentration in PBS were incubated at 37° C. for 1 hour. After 1 hour, the synthetic melanin was removed from the solution, and the relative concentrations of the compound left in solution (i.e., not bound to the melanin) were measured spectrophometrically. Table 1 below summarizes the relative binding affinity of various compounds to free synthetic melanin and is expressed as relative percentage of compounds of interest bound to the free synthetic melanin:

TABLE 1

| | % Melanin |
|---|---|
| Compound | Bound |
| Indole | 7.3 |
| 3-(2-Hydroxyethyl)indole | 6.5 |
| 3-Aminoquinoline | 5.1 |
| Cetylpyridinium chloride | 59.8 |
| Pyrocatechol | 21.5 |
| Niacinamide | 0 |
| Propylene Carbonate | Not detected |
| Thiodipropanoic Acid | 1.5 |
| Dilauryl Thiodipropionate | Not detected |
| Pyridine hydrochloride | 0 |
| 1-Butylpyridinium chloride | 0 |
| 4-Amino-1-dodecyl-pyridinium | 49.5 |
| chloride | |
| 1-(2-Hydroxyethyl) pyridinium | 0 |
| chloride | |
| Caffeine | 4.4 |
| Deoxy-d-glucose | Not detected |
| Caffeic acid | 0 |
| Quercetin | 99.7 |
| Pyrogallol | Not detected |
| Gallic acid | 0 |
| Chloroquine (positive control) | 48.6 |

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described therein will become apparent to those skilled in the art from the foregoing description. Such modifications are also intended to fall within the scope of the appended claims. All publications cited herein are incorporated by reference in their entirety.

1.-41. (canceled)

42. A method for targeted delivery of a benefit agent to areas of human integument having melanin deposits, comprising topically applying to said integument a composition comprising the reaction product of a benefit agent and compound capable of binding to melanin having the structure of Formula (I): 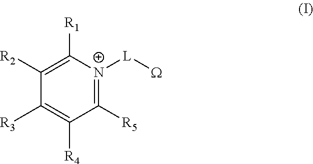
wherein, L is a divalent C₄₋₃₂ hydrocarbon moiety, optionally substituted with heteroatoms selected from Si, O, S, N, and halogen, and optionally substituted with one or more groups R; and is a functional group capable of forming a bond with said benefit agent; for conjugating said compound to a benefit agent, and
R₁-R₅ are independently selected, at each occurrence, from a group R; wherein R is selected from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —SH; —SR*; —C≡N, —NC; —(C═O)—R*; —CHO; —CO₂H; —CO₂⁻; —CO₂R*; —C(═O)Cl, —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃⁻; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; —CH₃, —CR*═CR*₂, —C≡CH, —Si(OR*)₃, —Si(R*)₂H, C₁-C₁₂ perfluoroalkyl; an aliphatic C₁-C₁₂ hydrocarbon radical; a C₁-C₁₂ aromatic hydrocarbon radical; or a C₁-C₁₂ heteroaryl radical;
R* is independently at each occurrence hydrogen or a straight chained, branched, or cyclic C₁-C₂₀ hydrocarbon radical, which may be saturated, partially saturated, or aromatic, each of which may be optionally substituted with 1-6 heteroatoms selected from nitrogen, oxygen, sulfur, or halogen, and wherein any two adjacent R* groups may together form a 3-8 membered aromatic, partially unsaturated, or saturated ring.
and topically acceptable salts thereof.

43. The method according to claim 42, wherein L is further characterized as having an octanol-water partition coefficient, expressed as Log(K_{ow}), of the corresponding alcohol HO-L-CH₃ between 1 and about 10.

44. The method according to claim 42, wherein L is a C₄₋₃₂ alkyl or alkenyl group.

45. The method according to claim 44, wherein L is a straight chained C₆₋₁₆ alkyl or alkenyl group.

46. The method according to claim 42, wherein L is a divalent C₄₋₃₂ hydrocarbon moiety of the form —X₁—(CR*₂)ₙ—X₂—(CR*₂)ₘ—X₃—, where X₁ is selected from the group consisting of a bond, —CH₂—, —CR*₂—, —CR*═CR*—, —C≡C—, —(C═O)—, —(C═O)—O—, —(C═O)—NR*—, —Si(R*)₂—, —(CR*₂)ᵢ—Si(R*)₂—, —(CR*₂)ᵢ—(Si(R*)₂)ⱼ—(O—Si(R*)₂)ₖ— (where “i” is an integer from 0-10, “j” is zero or one and “k” is an integer from 1-10); and X₂, and X₃ are independently at each occurrence a bond, —O—, —NR*—, —S—, —CH₂—, —CR*₂—, —CR*═CR*—, —C≡C—, —(C═O)—, —(C═O)—O—, —(C═O)—NR*—, —(OCH₂CH₂)_{y}—, or —(CH₂CH₂O)_{z}— (wherein “y” and “z” are independently an integer from 1 to 12), —Si(R*)₂—, —(Si(R*)₂)ⱼ—(O—Si(R*)₂)ₖ— (where “j” is zero or one and “k” is an integer from 1-10), and “n” and “m” are independently an integer from 0 to 30

47. The method according to claim 46, wherein “n” and “m” are independently integers from 6 to 24, with the proviso that at least one of “m” and “n” is not zero.

48. The method according to claim 46, wherein L has the form —(CH₃)_{z}— (where “z” is an integer from 6-18), —R^{a}—O—R^{b}—, —R^{a}—S—R^{b}—, —R^{a}—Si(CH₃)₂—R^{b}—, —R^{a}—Si(CH₃)₂—O—Si(CH₃)₂—R^{b}—, or —R^{a}—Si(CH₃)₂—(O—Si(CH₃)₂)ₖ—R^{b}—, where R^{a} and R^{b} are independently C₁₋₁₂ straight chain alkyl groups, and “k” is an integer from 1-12.

49. The method according to claim 42, wherein R₁, R₂, R₄, and R₅ are hydrogen.

50. The method according to claim 42, wherein Ω is selected from chloro, bromo, iodo, mesyl, tosyl, —OH, —SH, —NH₂; —NHR*, —(C—NR*₂)═R*, —CR*═CR*₂, —C≡CH, —CR*(O)CR* (epoxide), —Si(OR*)₃, —Si(R*)₂H, —(Si(R*)₂)ⱼ—(O—Si(R*)₂)ₖ—Si(OR*)₃, —(Si(R*)₂)ⱼ—(O—Si(R*)₂)ₖ—Si(R*)₂H, —CO₂H; —CO₂R*; —C(═O)Cl, —CH₂O(C═O)CH₂(C═O)CH₃, —C(O)—O—(N(—(C═O)—R*)₂), —SO₂Cl, —NCS, —NCO, —N₃, —N₂⁺Cl; or a group R.

51. The method according to claim 42, wherein Ω is a trialkoxysilane of the form —Si(OR*)₃ where R* is independently selected at each occurence from C₁₋₆ hydrocarbons.

52. The reaction product of an inorganic oxide particle and a compound of Formula (I): 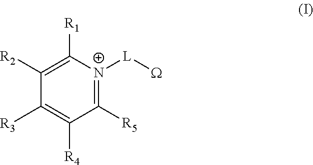
where L has the form —(CH₃)_{z}—, where “z” is an integer from 6-18;
Ω is a trialkoxysilane of the form —Si(OR*)₃, where R* is independently selected at each occurence from C₁₋₆ hydrocarbons; and
R₁, R₂, R₄, and R₅ are hydrogen, R₃ is hydrogen, —CH₃, —OCH₃, —OH, —SH, or —NH₂.

53. The method according to claim 52, wherein said inorganic oxide is selected from the group consisting of iron oxides, titanium dioxide, zinc oxide, alumina, and silica.

54. A surface-functionalized pigment, wherein said pigment has covalently attached to the surface thereof, a plurality lipophilic pyridinium compounds.

55. A compound of Formula (II): [chemical structure]
